(19) United States
(12) Patent Application Publication
(10) Pub. No.: US 2025/0339163 A1
(43) Pub. Date: Nov. 6, 2025

(54) INTRAVASCULAR LITHOTRIPSY CATHETER WITH OSCILLATING TIP

(71) Applicant: Shockwave Medical, Inc., Santa Clara, CA (US)

(72) Inventors: Khanh VO, Daly City, CA (US); Jonathan GALLEGO, San Jose, CA (US)

(73) Assignee: Shockwave Medical, Inc., Santa Clara, CA (US)

(21) Appl. No.: 18/653,178

(22) Filed: May 2, 2024

(51) Int. Cl. A61B 17/22; A61B 17/00

(52) U.S. Cl. CPC A61B 17/22022; A61B 17/00234; A61B 2017/00154; A61B 2017/00238; A61B 2017/00309; A61B 2017/0084; A61B 2017/00862; A61B 2017/00938; A61B 2017/22008; A61B 2017/22038; A61B 2017/22064

(57) ABSTRACT

Described herein are shock wave catheters and methods of use thereof for treating occlusions in a body lumen, the catheter comprising: a catheter enclosure; one or more shock wave emitters enclosed within the catheter enclosure; and an elongated body that extends to at least a distal end of the catheter enclosure, wherein a distal end of the elongated body is configured to vibrate from shock waves produced by the shock wave emitters to deliver mechanical forces to an occlusion in the body lumen.

### FIELD OF THE DISCLOSURE

The present disclosure relates generally to the field of medical devices and methods, and more specifically to shock wave catheter devices for treating lesions in body lumens, such as calcified lesions and occlusions in vasculature and kidney stones in the urinary system.

### BACKGROUND

A wide variety of catheters have been developed for treating calcified lesions, such as calcified lesions in vasculature associated with arterial disease. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than 10 atm), causing the balloon to expand in a vessel to push calcified plaques back into the vessel wall and dilate occluded regions of vasculature.

More recently, the technique and treatment of intravascular lithotripsy (IVL) has been developed, which is an interventional procedure to modify calcified plaque in diseased arteries. The mechanism of plaque modification is through use of a catheter having one or more acoustic shock wave generating sources located within a liquid that can generate acoustic shock waves that modify the calcified plaque. IVL devices vary in design with respect to the energy source used to generate the acoustic shock waves, with two exemplary energy sources being electrohydraulic generation and laser generation.

For electrohydraulic generation of acoustic shock waves, a conductive solution (e.g., saline) may be contained within an enclosure that surrounds electrodes or can be flushed through a tube that surrounds the electrodes. The calcified plaque modification is achieved by creating acoustic shock waves within the catheter by an electrical discharge across the electrodes. The energy from this electrical discharge enters the surrounding fluid faster than the speed of sound, generating an acoustic shock wave. In addition, the energy creates one or more rapidly expanding and collapsing vapor bubbles that generate secondary shock waves. The shock waves propagate radially outward and modify calcified plaque within the blood vessels. For laser generation of acoustic shock waves, a laser pulse is transmitted into and absorbed by a fluid within the catheter. This absorption process rapidly heats and vaporizes the fluid, thereby generating the rapidly expanding and collapsing vapor bubble, as well as the acoustic shock waves that propagate outward and modify the calcified plaque. The acoustic shock wave intensity is higher if a fluid is chosen that exhibits strong absorption at the laser wavelength that is employed. These examples of IVL devices are not intended to be a comprehensive list of potential energy sources to create IVL shock waves.

The IVL process may be considered different from standard atherectomy procedures in that it cracks calcium but does not liberate the cracked calcium from the tissue. Hence, generally speaking, IVL should not require aspiration nor embolic protection. Further, due to the compliance of a normal blood vessel and non-calcified plaque, the shock waves produced by IVL do not modify the normal vessel tissue or non-calcified plaque. Moreover, IVL does not carry the same degree of risk of perforation, dissection, or other damage to vasculature as atherectomy procedures or angioplasty procedures using cutting or scoring balloons.

More specifically, catheters to deliver IVL therapy have been developed that include pairs of electrodes for electrohydraulically generating shock waves inside an angioplasty balloon. Shock wave devices can be particularly effective for treating calcified plaque lesions because the acoustic pressure from the shock waves can crack and disrupt lesions near the angioplasty balloon without harming the surrounding tissue. In these devices, the catheter is advanced over a guide wire through a patient's vasculature until it is positioned proximal to and/or aligned with a calcified plaque lesion in a body lumen. The balloon is then inflated with conductive fluid (using a relatively low pressure of 2-4 atm) so that the balloon expands to contact the lesion but is not an inflation pressure that substantively displaces the lesion. Voltage pulses can then be applied across the electrodes of the electrode pairs to produce acoustic shock waves that propagate through the walls of the angioplasty balloon and into the lesions. Once the lesions have been cracked by the acoustic shock waves, the balloon can be expanded further to increase the cross-sectional area of the lumen and improve blood flow through the lumen. Alternative devices to deliver IVL therapy can be within a closed volume other than an angioplasty balloon, such as a cap, balloons of variable compliancy, or other enclosure.

When treating occlusions, a physician must first cross the occlusion (e.g., pass through the occluded area), and then feed the angioplasty balloon and/or other tools down the artery to the blockage to perform the desired procedure. In some instances, however, such as the case of a chronic total occlusion (“CTO”) or a resistant fibrotic lesion, the occlusion may be so tight and solid that it is difficult to cross the treatment device into the true lumen of the distal vessel. Conventional guide wires may have difficulty penetrating thick, fibrous lesions, and may risk trauma to blood vessels when navigating narrow and tortuous regions of vasculature. Some physicians may implement atherectomy procedures (e.g., laser-based, mechanically cutting or shaving, mechanically rotating devices, etc.) to form a channel in a lesion in combination with an angioplasty balloon treatment, but many atherectomy devices and systems carry a higher risk of vessel perforation or vessel dissection as compared with a basic angioplasty balloon catheters. Even if the initial puncture of a lesion is successful, placement of dilation devices, like angioplasty balloons, can be very difficult in chronically occluded vessels. This makes the treatment of resistant lesions a technically challenging procedure that requires a long learning curve for interventional cardiologists. Accordingly, there is an unmet need for a device that can penetrate and treat resistant fibrotic and calcified lesions, such as CTOs, while minimizing the risk of trauma to blood vessels. Similar devices are needed for treating occlusions formed in other parts of the body, for example, kidney stones in the urinary system.

### SUMMARY

An IVL catheter for delivering mechanical forces directly to an occlusion in a body lumen and methods of using an IVL catheter to apply mechanical forces to an occlusion are described herein. In some designs, the shock wave sources are positioned such that shock waves generated during IVL treatment impinge on an elongated body of the catheter. Based on the shock waves, the distal end of the elongated body can vibrate. If a guide wire is used during IVL treatment, the guide wire may vibrate in conjunction with the elongated body. The vibrating elongated body and optionally the guide wire may then be used to apply mechanical forces directly to an occlusion to penetrate and treat the occlusion.

In some aspects, a catheter for treating occlusions in a body lumen is provided, the catheter comprising: a catheter enclosure; one or more shock wave emitters enclosed within the catheter enclosure; and an elongated body that extends to at least a distal end of the catheter enclosure, wherein a distal end of the elongated body is configured to vibrate from shock waves produced by the shock wave emitters to deliver mechanical forces to an occlusion in the body lumen.

In some aspects, the distal end of the elongated body extends distally past the distal end of the catheter enclosure. In some aspects, the distal end of the catheter enclosure is sealed to the distal end of the elongated body. In some aspects the catheter enclosure comprises an angioplasty balloon. In some aspects, the catheter enclosure forms a closed volume around the elongated body. In some aspects, the catheter enclosure comprises an opening adjacent to one or more of the shock wave emitters. In some aspects, the opening is disposed in at least a tapered region of the catheter enclosure. In some aspects, the opening comprises a slit, the slit longitudinally aligned with a longitudinal axis of the elongated body. In some aspects, the elongated body comprises a guide wire lumen. In some aspects, the guide wire lumen is sized to receive a 0.014″ diameter guide wire. In some aspects, a diameter of the guide wire lumen is at least 0.0141″. In some aspects, vibration of the distal end of the elongated body causes a guide wire in the guide wire lumen to vibrate in conjunction with the distal end of the elongated body. In some aspects, the elongated body comprises a polymeric material. In some aspects, the elongated body comprises a first material, the catheter enclosure comprises a second material, and the first material is more rigid than the second material. In some aspects, the one or more shock wave emitters comprises an electrode pair. In some aspects, a first electrode of the electrode pair comprises a conductive sheath disposed around at least a portion of the elongated body. In some aspects, a second electrode of the electrode pair comprises a distal end of a conductive wire. In some aspects, the one or more shock wave emitters comprises an optical fiber. In some aspects, the one or more shock wave emitters comprises a first shock wave emitter and a second shock wave emitter. In some aspects, the second shock wave emitter is no greater than 90 degrees apart from the first shock wave emitter relative to a circumference of the elongated body. In some aspects, the second shock wave emitter is approximately 60 degrees apart from the first shock wave emitter relative to the circumference of the hollow tubular body.

In some aspects, a method of treating an occlusion in a body lumen is provided, the method comprising: inserting a catheter into the body lumen, the catheter comprising: a catheter enclosure; one or more shock wave emitters enclosed within the catheter enclosure; and an elongated body that extends to at least a distal end of the catheter enclosure; advancing the catheter within the body lumen until the distal end of the elongated body is positioned proximate to the occlusion; and applying energy to the one or more shock wave emitters to generate shock waves at the one or more shock wave emitters, wherein a distal end of the elongated body is configured to vibrate from the shock waves to deliver mechanical forces to the occlusion.

In some aspects, the catheter enclosure comprises an opening adjacent to one or more of the shock wave emitters. In some aspects, the opening is configured to open responsive to the generation of a shock wave, and the opening is configured to close after termination of a shock wave. In some aspects, applying energy to the one or more shock wave emitters comprises applying a voltage to one or more of the shock wave emitters. In some aspects, applying energy to the one or more shock wave emitters comprises applying a series of voltage pulses to one or more of the shock wave emitters. In some aspects, the series of voltage pulses are applied at a frequency between 4 Hz and 8 Hz. In some aspects, applying energy to the one or more shock wave emitters comprises applying laser energy to one or more of the shock wave emitters. In some aspects, the elongated body comprises a guide wire lumen, and inserting the catheter into the body lumen comprises: inserting a guide wire into the body lumen; and inserting the catheter into the body lumen over the guide wire. In some aspects, the vibration of the distal end of the elongated body causes the guide wire to vibrate in conjunction with the distal end of the elongated body such that the guide wire also delivers mechanical forces to treat the occlusion.

In some aspects, a system for treating occlusions in a body lumen is provided, the system comprising: a catheter comprising: a catheter enclosure; one or more shock wave emitters enclosed within the catheter enclosure; and an elongated body that extends to at least a distal end of the catheter enclosure, wherein a distal end of the elongated body is configured to vibrate from shock waves produced by the shock wave emitters to deliver mechanical forces to an occlusion in the body lumen; and an energy generator configured to deliver energy to one or more of the shock wave emitters to generate the shock waves.

In some aspects, the one or more shock wave emitters comprises one or more electrode pairs, and the energy generator is configured to deliver a voltage to the one or more shock wave emitters. In some aspects, the one or more shock wave emitters comprises one or more optical fibers, and the energy generator is configured to deliver laser energy to the one or more optical fibers.

### DESCRIPTION OF THE FIGURES

The invention will now be described, by way of example only, with reference to the accompanying drawings, in which:

FIG. 1 illustrates an exemplary system that includes a catheter, a power source, and a guide wire, showing the catheter being used to treat a chronic total occlusion (CTO) in a blood vessel, according to one or more examples of the present disclosure.

FIG. 2 illustrates a side view of a distal portion of an exemplary catheter, according to one or more examples of the present disclosure.

FIG. 3 illustrates a perspective view of an elongated body and emitter assembly of an exemplary catheter showing the position of two emitters relative to the circumference of the elongated body.

FIGS. 4A-4B illustrate perspective views of a distal portion of an exemplary open-system catheter having an enclosure with an opening, according to one or more examples of the present disclosure. FIG. 4A illustrates the exemplary catheter with the opening in a closed state.

FIG. 4B illustrates the exemplary catheter with the opening in an open state, such as during the generation of a shock wave.

FIG. 5 illustrates a perspective view of a distal portion of an exemplary closed-system catheter, according to one or more examples of the present disclosure.

FIG. 6 illustrates a cross-sectional view of an elongated body of an exemplary catheter showing the position of various lumens of the elongated body.

FIG. 7 illustrates the results of an experimental trial where an open-system catheter was used to treat a phantom surface made of a calcium mineral mimicking a calcified lesion.

FIG. 8 illustrates the results of an experimental trial where a closed-system catheter was used to treat a calcium phantom surface.

FIG. 9 illustrates a flowchart of an exemplary method of treating an occlusion in a body lumen using a catheter, according to one or more examples of the present disclosure.

### DETAILED DESCRIPTION

The following description is presented to enable a person of ordinary skill in the art to make and use the various embodiments and aspects thereof disclosed herein. Descriptions of specific devices, assemblies, techniques, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

Described herein are examples of shock wave generating catheters that include a vibrating elongated body and at least one shock wave emitter enclosed within an enclosure for generating shock waves to treat lesions in body lumens. The shock wave emitter may be positioned such that shock waves generated by the emitter cause a distal end of the elongated body to vibrate. During a shock wave treatment, the shock wave catheter can be advanced to a region of a body lumen that is proximate to an occlusion, such as a fibrotic or calcified occlusion or a chronic total occlusion (CTO). Optionally, the catheter is advanced until the elongated body (or a guide wire inserted through the elongated body) is in proximity to or in contact with the occlusion. Once positioned near the occlusion, energy can be applied by an energy source, such as a laser energy source or a voltage source, to generate shock waves at one or more emitters inside the enclosure. At least a portion of the shock wave energy is translated into mechanical movement (i.e., vibration) of the distal end of the elongated body. In some examples, repeated shock waves are generated, causing the distal end of the elongated body to vibrate. The elongated body can then be advanced into the occlusion to penetrate and mechanically disrupt the occlusion. When the catheter is used with a guide wire, the guide wire may move and vibrate in conjunction with the elongated body to deliver mechanical forces to the occlusion. Thus, the vibrating guide wire may be used to apply mechanical forces to treat the occlusion in addition to or in alternative to the mechanical forces applied by the vibrating elongated body of the catheter.

Advantageously, the use of mechanical forces from the elongated body and/or guide wire can increase the amount of force delivered to occlusion during a shock wave treatment, making IVL treatments quicker and more effective. The application of direct mechanical forces may allow users of the catheter to more easily penetrate and clear treatment-resistant lesions, such as calcified and fibrotic occlusion and CTOs, compared to conventional treatment methods. Further, the dynamic mode of action of the catheter, wherein the repeated generation of shock waves causes vibration of the elongated body and/or guide wire, allows users to continuously penetrate and drill into occlusions, streamlining IVL treatment. In some examples, openings may be provided in the enclosure of the catheter to allow cavitation bubbles to escape the enclosure and impinge on the occlusions, enhancing treatment of lesions near the catheter enclosure.

As used herein, the term “electrode” refers to an electrically conducting element (typically made of metal) that receives electrical current and subsequently releases the electrical current to another electrically conducting element. In the context of the present disclosure, electrodes are often positioned relative to each other, such as in an arrangement of an inner electrode and an outer electrode. Accordingly, as used herein, the term “electrode pair” refers to two electrodes that are positioned adjacent to each other such that application of a sufficiently high voltage to the electrode pair will cause an electrical current to transmit across the gap (also referred to as a “spark gap”) between the two electrodes (e.g., from an inner electrode to an outer electrode, or vice versa, optionally with the electricity passing through a conductive fluid or gas therebetween). In some contexts, one or more electrode pairs may also be referred to as an emitter assembly. In the context of the present disclosure, the term “emitter” broadly refers to the region of an emitter assembly where the current transmits across the electrode pair, generating a shock wave. The terms “emitter sheath” and “emitter band” refers to a continuous or discontinuous band of conductive material that may form one or more electrodes of one or more electrode pairs, thereby forming a location of one or more emitters.

One or more of the emitters (e.g., the electrodes thereof) may be formed from a metal, such as stainless steel, copper, tungsten, platinum, palladium, molybdenum, cobalt, chromium, iridium, or an alloy or alloys thereof, such as cobalt-chromium, platinum-chromium, cobalt-chromium-platinum-palladium-iridium, or platinum-iridium, or a mixture of such materials.

In some embodiments, an IVL catheter is a so-called “rapid exchange-type” (“Rx”) catheter provided with an opening portion through which a guide wire is guided (e.g., through a middle portion of a central tube in a longitudinal direction). In other embodiments, an IVL catheter may be an “over-the-wire-type” (“OTW”) catheter in which a guide wire lumen is formed throughout the overall length of the catheter, and a guide wire is guided through the proximal end of a hub.

Although the examples of shock wave devices described herein generate shock waves based on high voltage applied to electrodes, it should be understood that this disclosure encompasses shock wave devices that additionally or alternatively include a laser and optical fibers as a shock wave emitter system whereby the laser source delivers energy through an optical fiber and into a fluid to form shock waves and/or cavitation bubbles.

Shock wave catheters, according the principles described herein, can include various shock wave emitters in various configurations. For example, catheters have been developed that take advantage of the constructive interference that occurs between shock waves generated at closely-spaced shock wave emitters. In these catheters, the shock waves emitters are positioned such that shock waves generated at the emitters interfere to produce combined shock waves having greater shock wave energy than non-interfering shock waves. For instance, U.S. Patent Appl. No. 63/257,397, incorporated herein by reference in its entirety, provides examples of shock wave emitters configured to generate constructively interfering shock waves that can be used for shock wave catheters described herein. Efforts have also been made to direct acoustic energy from the shock waves in a forward direction to break up tighter and harder-to cross occlusions in vasculature. Examples of forward-firing emitter designs can be found in U.S. Pat. No. 10,966,737 and U.S. Publication No. 2019/0388110, both of which are incorporated herein by reference in their entirety. Such emitters may be used for any of the shock wave emitters described herein. Catheters have also been developed for delivering direct mechanical forces to lesions in conjunction with the generation of shock waves. For instance, shock wave catheters have been developed that include impactors that advance into lesions responsive to the generation of shock waves to deliver direct mechanical forces to a lesion. Features of such catheters that can be combined with the features of catheters described herein are described in U.S. Patent. Appl. No. 63/252,467 and U.S. patent application Ser. No. 18/513,421, incorporated herein by reference in their entirety.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof. As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement.

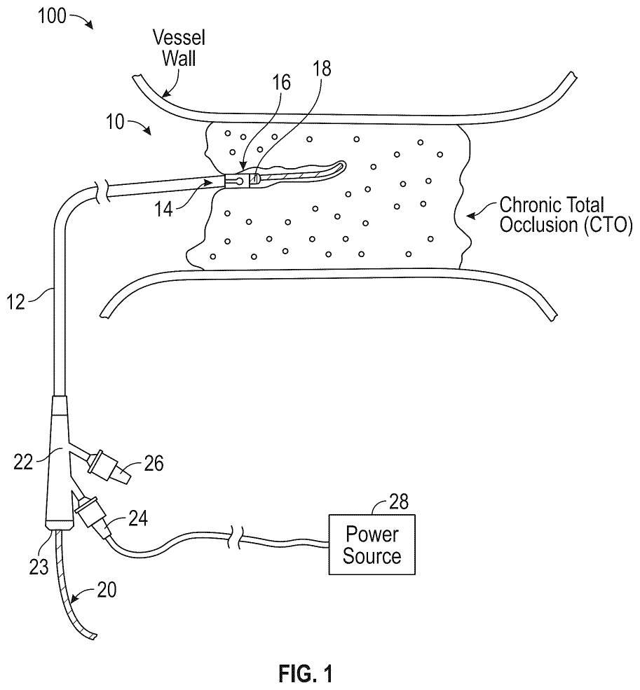

FIG. 1 depicts an exemplary catheter system 100 for treating lesions in a body lumen, such as the chronic total occlusion in the vessel wall pictured in FIG. 1. The system 100 includes a shock wave catheter 10, a power source 28, and a guide wire 20. The catheter 10 includes an elongated body 12 that extends distally from a handle 22 of the catheter 10. A distal portion of the elongated body 12 includes at least one enclosure 18 (e.g., an inflatable angioplasty balloon or a non-inflatable cap) and at least one shock wave emitter 16 (referred to below in singular form merely for simplicity) for generating shock waves inside the enclosure 18. A power source 28 is electrically connected to and configured for delivering energy pulses (e.g., one or more high-voltage pulses or laser energy pulses) to the at least one emitter 16 to generate shock waves at the emitter 16 inside the enclosure 18.

Generating shock waves at the emitter 16 may additionally cause a distal end 14 of the elongated body 12 to vibrate or oscillate, such that the elongated body 12 (and/or a guide wire 20 inserted through the catheter 10) can be used to deliver mechanical forces to a lesion. The enclosure 18 may include one or more openings proximate to the emitter 16, such as one or more skived openings or slits aligned with a longitudinal axis of elongated body 12. The opening may be configured to open responsive to the generation of a shock wave to allow cavitation bubbles formed by the shock waves to escape the enclosure 18, directing the cavitation bubbles to an occlusion. The opening may be configured to close after a shock wave terminates, and the opening may remain in a closed position when shock waves are not being generated by the emitter 16.

The enclosure 18 of the catheter 10 extends circumferentially around a portion of the elongated body 12 to surround the emitter 16 and at least a portion of the elongated body 12. The enclosure 18 may be sealed to a region of the elongated body 12 near the distal end 14 of the elongated body. The enclosure 18 may be filled with a fluid, such as a conductive fluid (e.g., saline), that allows electrical current to flow across the emitter 16 and acoustic shock waves formed at the emitter 16 to propagate within the enclosure 18. In some examples, the fluid may also contain an x-ray contrast fluid to permit fluoroscopic viewing of the catheter 10 and enclosure 18 by a surgeon during use. When filled with the fluid, the enclosure 18 may expand to provide an annular channel around the elongated body 12 that creates a space between the emitter 16 and the walls of the enclosure 18, minimizing the risk of damage to the enclosure 18 during a shock wave treatment. In a deflated state, the enclosure 18 may be positioned proximate to the elongated body 12 and, optionally, in a folded state, which may improve the maneuverability of the catheter 10 during insertion and positioning of the catheter 10. In some examples, the enclosure 18 is formed from a compliant or semi-compliant material. An example of a suitable material is an elastomeric polymer. In some examples, the enclosure 18 is a balloon, such as an inflatable angioplasty balloon, and the enclosure 18 expands when filled with fluid. In other examples, the enclosure 18 may be formed from a material that can be pressurized (e.g., pressurized by filling the enclosure 18 with fluid) without significant expansion (i.e., ballooning) of the enclosure 18.

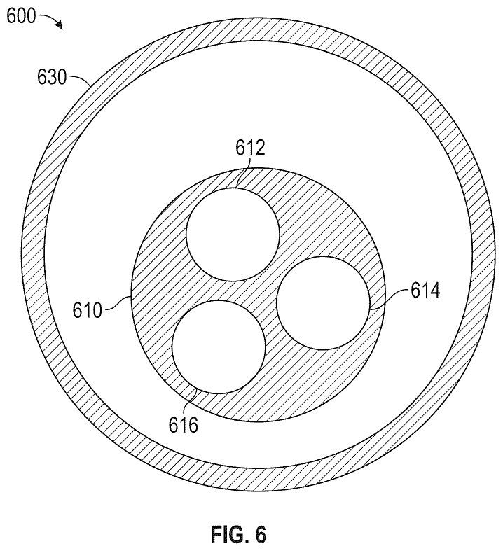

The elongated body 12 of the catheter 10 may include various lumens and/or channels sized for carrying fluid, conductive wires, and other components between the proximal handle 22 of the catheter 10 and a distal portion of the catheter 10, such as one or more fluid lumens for carrying fluid introduced through a fluid port 26 to the enclosure 18 and various conductive wires and/or optical fibers that enter the elongated body 12 through one or more wire ports 24 and carry energy from the power source 28 to the emitter 16. Various exemplary lumens of a catheter 10 are shown in FIG. 6, described in greater detail below. In some examples, a handle 22 of the catheter 10 includes a guide wire port. In such examples, a guide wire 20 may be inserted via a port of the catheter 10 (e.g., a port in the distal end of the catheter 10) and extended through a guide wire lumen of the elongated body 12 to aid in insertion and positioning of the distal end of the catheter 10. The guide wire 20 may exit at the proximal end of the catheter 10 through the guide wire port 23. However, in some examples the elongated body 12 does not include a guide wire lumen and the handle 22 does not include a guide wire port 23.

The distal end 14 of the catheter 10 is configured to be inserted into a body lumen of a patient, such as a blood vessel, a valve, or a ureter. The emitter 16 and enclosure 18 may be mounted near a distal end 14 of the elongated body 12 such that, when the catheter 10 is positioned in a body lumen, the emitter 16, enclosure 18, and distal end 14 of the elongated body 12 are proximate to a lesion targeted for treatment by the catheter 10. The elongated body 12 of the catheter 10 may be formed from one or more flexible materials, such that the distal end 14 of the elongated body 12 can flex during insertion, positioning, and removal of the catheter 10 and vibrate responsive to the generation of shock waves by the emitter 16. In some examples, the elongated body 12 is formed from one or more polymeric materials, such as polytetrafluoroethylene (PTFE, e.g., Teflon), polyether block amide (e.g., Pebax), nylon, urethane, or some other polymeric material.

To generate shock waves, high energy pulses (e.g., voltage or laser energy pulses) are applied to the emitter 16 by the external power source 28. In some examples, the emitter 16 of the catheter 10 may include at least one electrode pair formed from two closely-spaced electrodes, and the shock waves are generated by applying a voltage pulse to an electrode pair to cause current to flow across a spark gap between the electrodes of a pair. In such examples, the power source 28 is a voltage pulse generator (e.g., a four-kilovolt (4 kV) generator) that is configured for delivering electrical pulses to the at least one emitter 16. In some examples, the emitter 16 may be formed from one or more regions of conductive material (e.g., metal) that form the electrodes of an electrode pair. In a particular example, the emitter 16 of the catheter 10 is formed from a conductive sheath (e.g., a metal emitter band) mounted to the elongated body 12 and the conductive region of one or more wires placed in close proximity to the conductive sheath.

In some examples, the emitter 16 of the catheter 10 may be formed from the ends of optical fibers that extend along the elongated body 12 and terminate within the enclosure 18. In such examples, the power source 28 may be an energy pulse generator configured for delivering laser pulses to the emitter 16 via at least one optical fiber. Shock waves may be generated near the terminal ends of the optical fibers by delivering laser energy through the optical fibers and into the fluid within the enclosure 18.

Any desired number of emitters may be included in an exemplary shock wave catheter 10, such as one, two, three, four, five, six, eight, or more than eight emitters. The emitters (e.g., emitter 16 and any further emitters) may be arranged in a particular configuration along and/or around the distal portion of the elongated body 12. For example, an emitter 16 may be located proximate to the distal end 14 of the elongated body 12 and configured for generating shock waves that impinge on the distal end 14 of the elongated body 12 to cause the distal end 14 to vibrate. In other examples, two or more emitters may be spaced apart along a length of the elongated body 12. For instance, various emitters may be arranged on the elongated body 12 in groupings, such as a proximal set of emitters, a medial set of emitters, and a distal set of emitters. In some examples, two or more emitters are wired together (e.g., wired in series and/or in parallel) such that the emitters generate shock waves together when activated by the power source 28 (e.g., when a voltage pulse or laser pulse is delivered by the power source 28). In some examples, various emitters may be wired separately (e.g., wired on separate circuits), such that a particular emitter (e.g., emitter 16) or subset of emitters can be selectively activated by the power source 28 to generate shock waves.

To operate the catheter 10, a physician optionally positions the catheter 10 over the end of a guide wire 20 such that the guide wire 20 extends through the elongated body 12 of the catheter 10. The physician may then insert the catheter 10 into a body lumen and advance the catheter 10 over the guide wire 20 until the distal end 14 of the elongated body 12 is positioned proximate to an occlusion in the body lumen. The physician can track the position of the guide wire 20 and catheter 10 within a patient by use of real-time and/or static imaging devices, including x-ray imaging, intravascular ultrasound (IVUS), optical coherence tomography (OCT), radiofrequency (RF) navigation, and other such techniques.

When the distal end 14 of the catheter 10 has been positioned near a lesion in the body lumen, the enclosure 18 can be filled with a conductive fluid through the fluid port 26, optionally such that the enclosure 18 expands to contact the wall of the body lumen and/or a lesion. The power source 28 is then used to deliver one or more high voltage pulses or laser pulses to the emitter 16 to create shock waves within the enclosure 18. The shock waves propagate within the enclosure 18 and impinge on the distal end 12 of the elongated body 12, causing the distal end to vibrate within the body lumen to deliver mechanical forces directly to a lesion. The shock waves may additionally propagate outwardly from the emitter 16 and toward the inner surface of the enclosure 18, through the material of the enclosure 18, and into a lesion in a body lumen proximate to the enclosure 18 where the energy may at least partially disrupt the lesion. In some examples, cavitation bubbles formed by the shock waves may exit the enclosure 18 through an opening in the enclosure 18, causing the bubbles to be directed into a lesion to apply additional force to the lesion. During a shock wave treatment, a series of shock waves can be generated to cause repeated delivery of shock wave energy to the elongated body 12 and to lesions proximate the enclosure 18. In some examples, the generation of a series of shock waves causes the distal end 14 of the elongated body 12 to vibrate or oscillate such that the distal end 14 delivers repeated mechanical forces to penetrate and tear lesion.

In some examples, the magnitude of the shock waves can be controlled by controlling the magnitude, current, duration, and/or repetition rate of the power supplied by the power source 28. The preferred voltage, repetition rate, and number of pulses may vary depending on, e.g., the size of the lesion, the extent of calcification, the size of the blood vessel, the attributes of the patient, or the stage of treatment. In some examples, the magnitude of power delivered by the power source 28 may be adjusted during the course of a shock wave treatment. For instance, a physician may start with low energy shock waves and increase the energy as needed to disrupt and clear the lesion (or vice versa). Further, in examples where one or more emitters 16 are wired on separate circuits or separate circuit branches to be operated separately, a physician may selectively emit shock waves at only a particular subset of emitters by applying energy to only that subset of the emitters. For instance, a physician may first generate shock waves at a first subset of the emitters (e.g., a distal subset of emitters that includes at least emitter 16) to cause the distal end of the elongated body 12 and the guide wire 20 to vibrate, and may continue treatment by generating shock waves at a second subset of emitters (e.g., a medial or proximal subset of emitters) to treat lesions surrounding the enclosure 18. After a first series of one or more shock waves are delivered, the catheter 10 can be repositioned or advanced further in the body lumen to continue treatment.

For treatment of an occlusion in a blood vessel, a voltage pulse applied by the power source 28 is typically in the range of from about five hundred to three thousand volts (500 V-3,000 V). In some implementations, the voltage pulse applied by the power source 28 can be up to about ten thousand volts (10,000 V) or higher than ten thousand volts (10,000 V). The pulse width of the applied voltage pulses can range between two microseconds and six microseconds (2-6 μs). The repetition rate or frequency of the applied voltage pulses may be between about 1 Hz and 10 Hz. The total number of pulses applied by the power source 28 may be, for example, sixty (60) pulses, eighty (80) pulses, one hundred twenty (120) pulses, three hundred (300) pulses, up to five hundred (500) pulses, or other increments of pulses within this range. Alternatively or additionally, in some examples, the power source 28 may be configured to deliver a packet of micro-pulses having a sub-frequency between about 100 Hz-10 kHz.

The progress of the procedure may be monitored by one or more of the imaging techniques described above. As the lesion is broken up or penetrated by mechanical forces from the vibrating elongated body 12 and/or guide wire 20, the guide wire 20 and catheter 10 can be advanced farther into the lesion, and the shock wave treatment can be repeated until the total occlusion is cleared or until the diameter of the vessel permits the placement of a second treatment device having a larger profile. For example, the enlarged channel can receive a different catheter having a more conventional angioplasty balloon or differently oriented shock wave sources. Catheters of this type are described in U.S. Pat. No. 8,747,416 and U.S. Publication No. 2019/0150960, cited above. Once the lesion has been sufficiently treated, the catheter 10 and the guide wire 20 can be withdrawn from the body lumen.

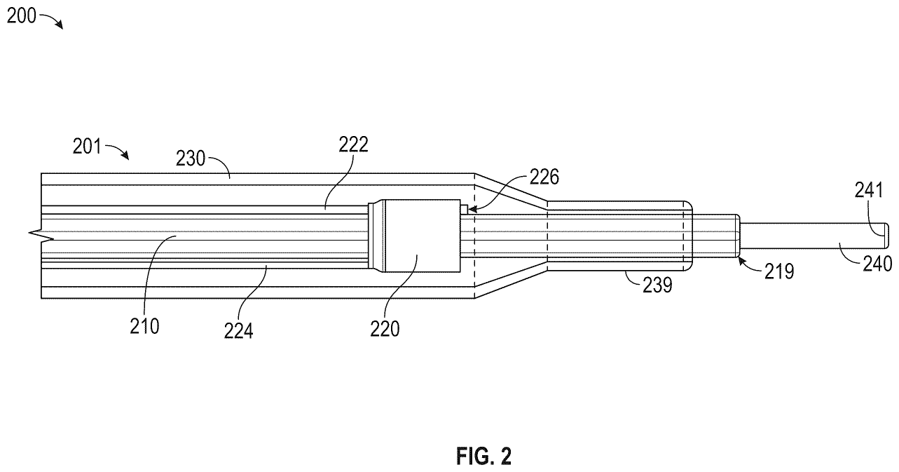

As described above, a distal portion of the catheter 10 can be inserted into a patient's body lumen and includes elements of the catheter 10 that can be used to treat a lesion during a shock wave treatment. FIG. 2 illustrates the distal portion 201 of an exemplary catheter 200 that can be used for catheter 10, the catheter 200 including a catheter enclosure 230, one or more shock wave emitters 226 enclosed within the enclosure 230, and an elongated body 210 that extends to at least a distal end 239 of the enclosure 230. As described above, the elongated body 210 may be configured to vibrate based shock waves produced by one or more shock wave emitters 226. In some examples, the elongated body 210 may absorb at least some of the shock wave energy generated by the one or more shock wave emitters 226. Vibration of the shock wave may include at least the distal end 219 of the elongated body 210 moving in a direction away from the emitter 226. Repeated generation of shock waves may cause the vibration of the distal end 219 of the elongated body 210 to deliver mechanical forces to treat occlusions in the body lumen. During a shock wave procedure, a user may maneuver the elongated body 210 into an occlusion to repeatedly deliver mechanical forces to penetrate and clear the occlusion.

In some examples, the distal end 219 refers to the length of the elongated body 210 that extends past (i.e., more distally than) the location of the emitter 226. In some examples, only the distal end 219 of the elongated body 210 vibrates. Accordingly, more proximal portions of the elongated body 210 may not vibrate responsive to the generation of shock waves or may vibrate relatively less than the vibration of the distal end 219. In some examples, the length of the distal end 219 is greater than one millimeter (1 mm), greater than two millimeters (2 mm), or greater than three millimeters (3 mm). In some examples, and as shown in FIG. 2, the length of the distal end 219 is between three millimeters (3 mm) and three and a half millimeters (3.5 mm).

The catheter 200 of FIG. 2 is illustrated with a guide wire 240 extending through the elongated body 210. As mentioned above, a user of the catheter 200 may insert and position the catheter inside the body lumen with aid from a guide wire 240 extended through the elongated body 210 of the catheter 200. In some examples, the guide wire 240 is a commercially available guide wire used for angioplasty procedures (e.g., a 0.35 mm, or 0.014″ diameter guide wire). However, in some examples, the guide wire 240 is modified. For instance, a distal tip of the guide wire 240 may be removed by cutting the distal end 241 of the guide wire 240. Additionally or alternatively, features may be added to the distal end 241 of the guide wire 240 to improve the delivery of mechanical forces to the occlusion or reduce the risk of harm to the walls of the body lumen. Such features may include one or more of a modified guide wire tip, a cap for the guide wire tip, or shaped features that improve delivery of mechanical force by the guide wire 240.

In some examples, the guide wire 240 may remain inside the elongated body 210 during a shock wave treatment such that the guide wire 240 vibrates in conjunction with the distal end 219 of the elongated body 2120 when shock waves are generated at the emitter 226. In some examples, a distal end 241 of the guide wire 240 may extend passed (i.e., more distally than) the distal end 219 of the elongated body 210. Accordingly, the distal end 241 of the guide wire 240 may be used to deliver mechanical forces to an occlusion in addition to or in alternative to the distal end 219 of the elongated body 210.

Components of the exemplary catheter 200 may be disposed around the circumference of elongated body 210, which forms a central shaft of the distal portion 201 of the catheter 200. The elongated body 210 may be formed of a material that is sufficiently flexible to allow the distal portion 201 of the catheter 200 to be navigated through body lumens, such as tortuous regions of a patient's vasculature or other body lumens. Furthermore, the material of the elongated body 210 may be sufficiently flexible to allow for the distal end 219 of the elongated body 210 to vibrate responsive to shock waves, while being resilient enough to avoid damage during a shock wave treatment. In some examples, the material of the elongated body 210 may be configured to absorb a portion of the shock wave energy produced by the emitter 226 and translate the shock wave energy into mechanical movement of the distal end 219 of the elongated body 210. In some examples, a first region of the elongated body 210 is formed from a first material, and a second region of the elongated body 210 is formed from a second material different from the first material. For instance, a distal portion of the elongated body 210 (e.g., the distal end 219 or a portion including the distal end 219 of the elongated body 210) may be formed from a relatively more flexible material than a proximal portion of the elongated body 210. Such a configuration may advantageously increase the magnitude of vibration of the distal end 219 of the elongated body 210 without sacrificing the structural stability of more proximal portions of the elongated body 210.

In some examples, grooves are formed in the outer surface of the elongated body 210. The grooves may extend longitudinally along the surface of the elongated body 210 and provide space for wires, lumens, and other components to extend along and be at least partially recessed into the outer surface of the elongated body 210. In some examples, the grooves are spaced evenly around the circumference of the elongated body 210. In various examples, the elongated body 210 may include two grooves, three grooves, four grooves, six grooves, eight grooves, ten grooves, or twelve grooves. In a particular example, and as shown in FIG. 2, the elongated body 210 includes six grooves spaced evenly around the circumference of the elongated body 210 (i.e., spaced at 60 degree increments around the circumference).

As described above, the elongated body 210 may further include one or more lumens for carrying fluid, power, and components of a catheter system from a proximal end of the catheter 200 to a distal end of the elongated body. For instance, the elongated body 210 may include a guide wire lumen for carrying a guide wire 240, one or more fluid lumens for flowing fluid from a fluid source into and out of the enclosure 230, and/or one or more wire lumens for carrying wires 222, 224 or optical fibers for delivering energy from a power source to the emitter 226. In some examples, the lumens are channels that extend longitudinally through the material of the elongated body 210. However, in other examples, the lumens may be configured as tubes extending along an outer surface of the elongated body 210 (e.g., in grooves formed in the outer surface). Various lumens of the elongated body 210 are described in further detail with respect to FIG. 6, below.

An enclosure 230 surrounds at least a portion of the elongated body 210, forming a closed volume around the elongated body 210 that encloses the emitter 226. During a shock wave treatment, the enclosure 230 may be filled with a fluid, such as saline or another conductive fluid. In some examples, fluid is continuously flushed through the enclosure 230 during a shock wave treatment to remove debris and bubbles formed from the generation of shock waves at the emitter 226. In some examples, the fluid enters the enclosure 230 via a fluid lumen, such as a lumen extending through the elongated body 210, or a lumen extending along a surface of the elongated body 210. The fluid may exit the enclosure 230 via an opening in the enclosure 230 or via a lumen of the catheter 200. In some examples, filling the enclosure 230 with fluid causes the enclosure to inflate (i.e., increase in diameter) such that the enclosure 230 can be inflated to contact the walls of a body lumen (and/or a lesion in the body lumen) during a shock wave treatment. In a particular example, the enclosure 230 may be an inflatable angioplasty balloon, such as a commercially available angioplasty balloon. When filled with fluid, the diameter of the enclosure 230 may provide a space between the emitter 226 and the inner surface of the enclosure 230, such that shock waves generated at the emitter 226 do not cause damage to the enclosure 230. In some examples, the enclosure 230 is inflatable by a relatively lesser amount when filled with fluid, or may not inflate when filled with fluid. For instance, the enclosure 230 may be formed from a relatively more rigid material, such as a rigid or semi-compliant polymeric material.

The enclosure 230 may be sealed (e.g., heat-sealed) to the elongated body 210 at one or more of its ends, such as at a distal end or at a proximal end of the enclosure 230. For example, FIG. 2 illustrates an enclosure 230 with its distal end 239 sealed to the distal end 219 of the elongated body 210. In some examples, the material of the enclosure 230 is the same as the material of the elongated body 210. In such examples, sealing (e.g., heat sealing) the enclosure 230 to the elongated body 210 may form a region of uniform material. In some examples, the material of the enclosure 230 may be different from the material of the elongated body 210. For instance, the elongated body 210 may be formed from a first material, the enclosure 230 may be formed from a second material. The first material may be more rigid than the second material, such that the material of the enclosure 230 is more flexible than the elongated body 210 (e.g., to permit inflation of the enclosure and/or improve robustness of the elongated body). As described above, a distal end 239 of the enclosure 230 may be sealed to the elongated body 210 such that the distal end 219 of the elongated body 210 includes material of both the elongated body 210 and the enclosure 230. In such examples, both the distal end 239 of the enclosure 230 and the distal end 219 of the elongated body 210 may vibrate responsive to the generation of shock waves to deliver mechanical forces to treat occlusion in a body lumen. In some examples, a distal end 219 of the elongated body 210 extends past a distal end 239 of the enclosure 230.

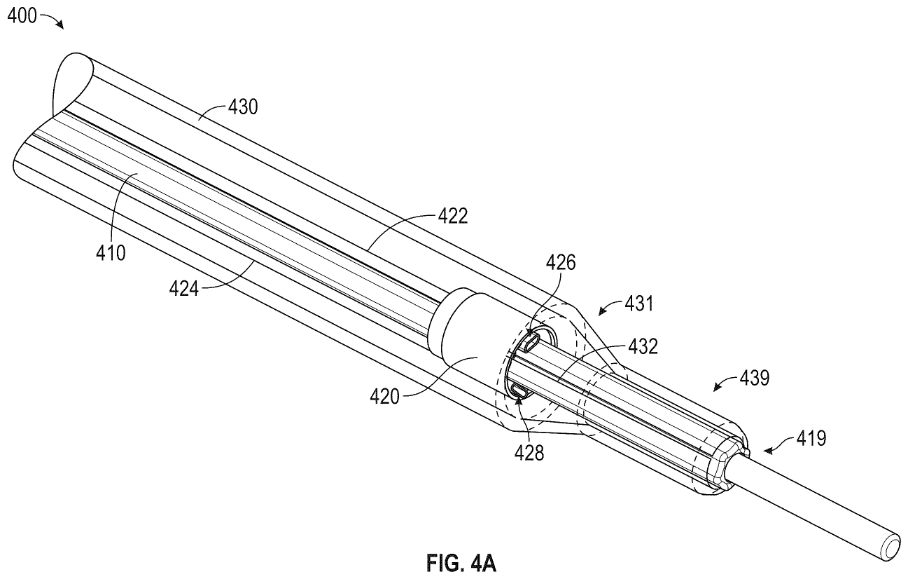

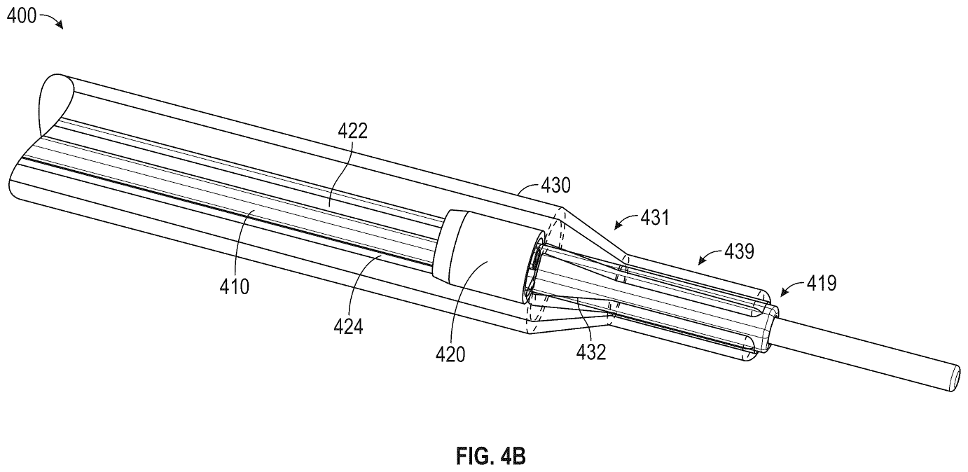

Optionally, the enclosure 230 includes one or more openings, such as slits or skived openings near the distal end 239 of the enclosure 230. For example, the one or more openings may be disposed at least partially in a tapered region of the enclosure 230. The openings may be configured to selectively open responsive to the generation of shock waves, and may close following termination of a shock wave. The one or more openings may be adjacent to one or more of the emitters 226. Various exemplary openings of the enclosure 230 are described in further detail with respect to FIGS. 4A-4B, below. In some examples, the enclosure 230 does not include openings.

The exemplary catheter 200 includes an emitter assembly that forms one or more emitters 226 of the catheter 200. Components of the emitter assembly may be mounted along an outer surface of the elongated body 210 and positioned such that the emitter 226 generates shock waves inside the enclosure 230. The emitter 226 may be configured to generate shock waves toward a distal end 219 of the elongated body 210 to facilitate vibration of the shock waves by the distal end 219 of the elongated body 210. In some examples, the emitter 226 includes at least one electrode pair and components (e.g., wires) to create one or more electrode pairs inside the enclosure 230. An electrode pair may be formed by two regions of conductive material separated by a small gap (i.e., a “spark gap”) across which current can flow to generate a shock wave. In such examples, a shock wave can be formed at the emitter 226 by applying a voltage to one or more electrodes of the electrode pair to create a potential difference across the electrode of the pair that causes current to flow between the electrodes. In some examples, the emitter 226 includes at least one optical fiber, and shock waves may be formed at the emitter 226 by applying laser energy to the at least one optical fiber.

As shown in FIG. 2, an exemplary emitter assembly may include at least a conductive sheath 220, a first wire 222, and a second wire 224. The conductive sheath 220 may be formed from an electrically conductive material, such as a metal (e.g., stainless steel, nickel, titanium, tungsten, platinum, palladium, molybdenum, or an alloy thereof). In some examples, the conductive sheath 220 is disposed around at least a portion of the elongated body 210 and may be fastened to an outer surface of the elongated body 210. The conductive sheath 220 may fit tightly around the elongated body 210 to secure the conductive sheath 220 to the elongated body 210. In some examples, the conductive sheath 220 encircles at least a portion of the circumference of the elongated body 210. In some examples, the conductive sheath 220 is continuous (i.e., cylindrically shaped or ring shaped), such that the conductive sheath 220 encircles the entire circumference of the elongated body 210. However, in other examples the conductive sheath 220 is discontinuous (i.e., encircling only a portion of the circumference of the elongated body 210).

The first wire 222 and second wire 224 of the emitter assembly may extend along an outer surface of the elongated body 210. Optionally, the wires 222, 224 extend within grooves in the outer surface of the elongated body 210. In some examples, the first wire 222 is a live wire (i.e., a wire that is connected to a positive or negative voltage terminal of a power source, such as the exemplary power source 28 shown in FIG. 1). In some examples, the second wire 224 is a return wire that is connected to ground. The first wire 222 and the second wire 224 of the emitter assembly may be commercially available wires, such as insulated wires that include a conductive interior formed of copper or another conductive metal. In some examples, the first wire 222 and/or second wire 224 are modified such that the distal ends of the wires 222, 224 are conductive. For instance, the distal end of the wires 222, 224 may be modified by removing insulation from the distal end of the wire or modified with the inclusion of additional conductive material at the distal end of the wire.

In the emitter 226 shown in FIG. 2, the second wire 224 can be coupled to the conductive sheath 220. Accordingly, a first electrode of an electrode pair may be formed by a region of the conductive sheath 220 (coupled to the wire 224), and a second electrode of the electrode pair may be formed from a conductive portion of a wire 222 proximate to the conductive sheath 220. As shown in FIG. 2, the distal end of the first wire 222 may extend past a distal edge of the conductive sheath 220, such that shock waves form in a spark gap between the distal end of the first wire 222 and the distal edge of the conductive sheath 220. Such a configuration may advantageously cause shock waves formed at the emitter 226 to propagate in a forward (i.e., distal) direction toward the distal end 219 of the elongated body 210. Various other emitter configurations are also envisioned for forming forward-firing emitters of a catheter 200 and described herein. Although not explicitly illustrated, in some examples, the catheter 200 may include one or more radially-firing emitters. For example, the catheter 200 may include one or more radially-firing emitters disposed within the elongated body 210 proximal to the emitter 226.

In some examples, one or more of the wires 222, 224 may be directly electrically connected to the conductive sheath 220 (e.g., by layering, crimping, soldering), such that current can flow between the wire and the conductive sheath 220 without traversing a spark gap and generating a shock wave. In the catheter 200 shown in FIG. 2, a conductive region of the second wire 224 is in contact with the conductive sheath 220 to provide a direct electrical connection. Accordingly, when a voltage is applied across the first wire 222 and the second wire 224 (e.g., a voltage applied by the power source 28 shown in FIG. 1), current may flow from the first wire 222 to the conductive sheath 220 across the spark gap, generating a shock wave at the emitter 226. The current may then flow from the conductive sheath 220 to the second wire 224 (without generating a shock wave) and to ground.

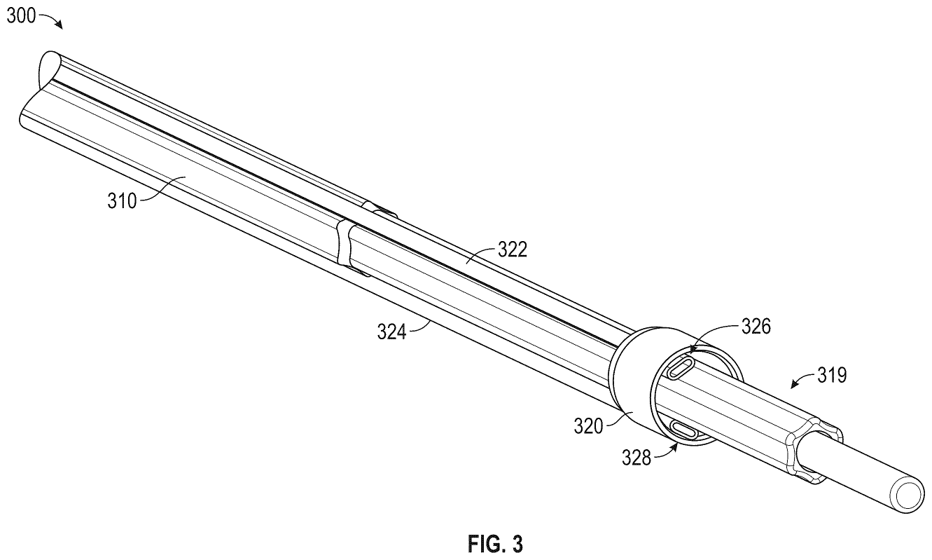

While the emitter assembly of the catheter 200 shown in FIG. 2 includes a single emitter 226, a catheter according to the present disclosure may include any number of emitters. For instance, FIG. 3 illustrates a perspective view of the elongated body 310 of an exemplary catheter 300 that can be used for catheter 10 in system 100, the catheter 300 including an emitter assembly that includes two emitters 326, 328 formed from a conductive sheath 320, a first wire 322, and a second wire 324. Optionally, the catheter 300 can include a guide wire 340 extending therethrough. The emitter assembly may be generally similar to the emitter assembly shown in FIG. 2, however the distal end of the second wire 324 shown in FIG. 3 is spaced apart from the conductive sheath 320 to form a second emitter 328 at a spark gap between the second wire 324 and the conductive sheath 320. Accordingly, when a voltage is applied across the first wire 322 and the second wire 324 in the emitter assembly of FIG. 3, current may flow from the first wire 322 to the conductive sheath 320 across a first spark gap, generating a first shock wave at the first emitter 326. The current may then flow from the conductive sheath 320 to the second wire 324 across a second spark gap, generating a second shock wave at the second emitter 328. The current may then flow through the second wire 324 and to ground.

As mentioned above, at least a portion of the shock wave energy impinges on the elongated body 310, causing at least the distal end 319 of the elongated body 310 to move in response to the generation of shock waves. To promote movement of the distal end 319 in a particular direction, two or more emitters 326, 328 may be positioned on a same side of the elongated body 310 (e.g., along a same portion of the circumference of the elongated body 310), such that concurrent shock waves generated by the emitters impinge on a same side of the elongated body 310. Such a configuration may cause an increased amount of force to be applied to a particular side of the elongated body 310 by the shock waves, to force the elongated body 310 in a direction away from the location of the first emitter 326 and second emitter 328 relative to the circumference of the elongated body 310. When more than one emitter (e.g., emitter 326, 328) is disposed on a same side of the elongated body 310, increased shock wave energy may impinge on the elongated body 310 compared to catheters 300 including a single emitter, or catheters including multiple emitters positioned on opposite sides of the elongated body 310. The shock waves generated by simultaneous firing of the first emitter 326 and second emitter 328 may therefore produce greater vibration of the distal end 219 and increased mechanical forces that can be applied to a lesion.

As shown in FIG. 3, in some examples the second emitter 328 may be positioned no greater than 180 degrees apart from the first emitter 326 relative to a circumference of the elongated body 310. In some examples, the second emitter 328 is positioned no greater than 90 degrees, or no greater than 45 degrees apart from the first emitter 326 relative to a circumference of the elongated body 310. In some examples, the location of the emitters 326, 328 is based on the location of grooves in the outer surface of the elongated body 310. For instance, the first wire 322 may extend longitudinally along the elongated body 310 along a first groove and form a first emitter 326, and the second wire 324 may extend longitudinally along the elongated body 310 along a second groove and form the second emitter 328. Accordingly, the position of the emitters 326, 328 relative to the circumference of the elongated body 310 may be based at least partially on the spacing of the grooves. In a particular example, the second emitter 328 is approximately 60 degrees apart from the first emitter 326 relative to the circumference of the elongated body 310. In various examples, the second emitter 328 may be approximately 15 degrees, approximately 30 degrees, approximately 45 degrees, approximately 75 degrees, or approximately 90 degrees apart from the first emitter 326 relative to the circumference of the elongated body 310.

In some examples, additional emitters may be included in an exemplary catheter 300. For instance, additional emitters may be formed by one or more additional conductive sheaths, one or more additional wires, and/or one or more additional optical fibers included in a catheter 300. In some examples, the additional emitters may be disposed on the elongated body 310 proximal to the emitters 326, 328. As noted above, in some examples, these proximally located emitters may be radially-firing shock wave emitters. Shock waves generated in a more proximal portion of the enclosure may be used to treat occlusions around a proximal portion of the enclosure. In some examples, shock waves generated at the emitters do not cause the distal end 319 of the elongated body 310 to vibrate (or may cause the elongated body 310 to vibrate relatively less than more distally positioned emitters, such as emitters 326, 328).

Returning to FIG. 2, when shock waves are generated at the emitter 226, the shock waves generate acoustic pressure waves that propagate outwardly through fluid inside the enclosure 230 of the catheter 200. As described above, at least a portion of the shock wave impinges on the elongated body 210 to cause the elongated body 210 (and, optionally a guide wire 240 inserted in the elongated body 210) to vibrate to apply mechanical forces to treat an occlusion. At least a portion of the shock wave may also propagate toward an enclosure 230 of the catheter 200 and through the walls of the enclosure 230 to treat lesions in the body lumen proximate to the surface of the enclosure 230. In some examples, the generation of shock waves may also produce cavitation bubbles when current flows across the emitter 226 through the fluid inside the enclosure 230. The expansion and bursting of the cavitation bubbles may create further acoustic energy in the fluid that can impinge on the elongated body 210, adding to the vibration of the distal end 219 of the elongated body 210 and/or the guide wire 240. Additionally or alternatively, acoustic energy form the cavitation bubbles may impinge on the inner surface of an enclosure 230, causing the acoustic energy to be transmitted through the enclosure 230 and into lesions proximate to the enclosure 230.

In some examples, the enclosure 230 includes one or more openings that allow at least a portion of the cavitation bubbles to escape the enclosure and enter the body lumen. Such a catheter may be referred to as an open system catheter and may allow for fluid communication between the volume inside the enclosure 230 and a body lumen in which the catheter 200 has been positioned. Advantageously, providing an opening in the enclosure 230 may allow for the formation of larger cavitation bubbles compared to cavitation bubbles formed in an enclosure 230 that lacks an opening. The expansion and bursting larger cavitation bubbles may cause greater acoustic forces to impinge on the elongated body 210 of the catheter, resulting in more intense movement and vibration of the distal end 219 and guide wire 240 and greater mechanical forces can be applied to occlusions during a shock wave treatment.

FIGS. 4A-4B illustrate an exemplary catheter 400 that includes an opening 432 in the enclosure 430 and can be used for catheter 10 in system 100 described above. The catheter 400 may be generally similar to the catheters 10, 200 and 300 described with respect to FIGS. 1, 2, and 3 above. For example, the catheter 400 may include an elongated body 410, an enclosure 430, a first wire 422 and a second wire 424 extending along the elongated body 410, and a conductive sheath 420 disposed around at least a portion of the elongated body 410. Optionally, the catheter 400 can include a guide wire 440 extending therethrough. The conductive sheath 420, first wire 422, and second wire 424 may form one or more emitters, such as the first emitter 426 and a second emitter 428.

In some examples, the opening 432 is a slit formed in a region of the enclosure 430. The slit may extend longitudinally along a length of the enclosure 430 and may be aligned with a longitudinal axis of the elongated body 410. In some examples, the opening 432 is disposed in at least a tapered region 431 of the enclosure 430. In some examples, the opening 432 terminates at a distal end 439 of the enclosure 430. In some examples, the opening 432 extends at least about 1 mm proximal to the conductive sheath 420. In some examples, the opening 432 extends at least about 1 mm distal to the conductive sheath 420. In some examples, the length of the opening 432 is about 3 mm, which can advantageously prevent unintentional tearing of the opening 432. In some examples, the opening 432 may be formed by skiving the material of the enclosure 430 (e.g., removing a small amount of the material to forming a slit in the enclosure). In some examples, the opening 432 may be configured to selectively open in response to the generation of shock waves at one or more of the emitters 426, 428. For instance, the narrow shape of the opening 432 and/or the compliant material properties of the enclosure 430 may cause the opening 432 to remain in a closed state when the enclosure 430 is filled with fluid and shock waves are not being generated at the emitters 426, 428. FIG. 4A illustrates the opening 432 in a closed state. When the opening 432 is in a closed state, the enclosure 430 may form a sealed volume around the elongated body 410, such that fluid in the elongated body 410 is not in fluid communication with the volume outside the enclosure 430. When a shock wave is generated at one or more of the emitters 426, 428, the opening 432 may be configured to open, thereby permitting fluid and cavitation bubbles to escape the enclosure 430. FIG. 4B illustrates the opening 432 in an open state.

As shown in FIGS. 4A-4B, the opening 432 in the enclosure 430 may be adjacent to one or more of the emitters 426, 428. In some examples, the opening 432 may be positioned such that the cavitation bubbles formed at the emitters 426, 428 are directed out of the enclosure 430 and toward a lesion in the body lumen. In some examples, the opening 432 is positioned outward from one or more of the emitters 426, 428 relative to a longitudinal axis of the elongated body 410, thereby directing the cavitation bubbles outward and toward lesions surrounding the enclosure 430. In some examples, the opening 432 is positioned distal to one or more of the emitters 426, 428, such that cavitation bubbles are directed in a forward direction and toward occlusions near the distal end 419 of the elongated body 410. In some examples, the opening 432 may be positioned between (e.g., at approximately an equal distance from) a first emitter 426 and a second emitter 428 relative to a circumference of the elongated body 410 and/or enclosure 430.

While the catheter 400 illustrated in FIGS. 4A-4B include a single opening 432 in the enclosure 430, in some examples a catheter may include a plurality of openings, such as a plurality of openings evenly spaced around a circumference of the enclosure 430. In a particular example, a number of openings may be equal to the number of emitters 426, 428 in a catheter 400 or, in particular, the number of emitters used to vibrate the distal end 419 of the elongated body 410 of the catheter. For example, a catheter 400 may include an opening 432 adjacent to each emitter 426, 428 of the catheter 400, or one or more openings 432 positioned at some other location relative to each emitter 426, 428 (e.g., a distal direction and/or outward direction relative to each emitter 426, 428).

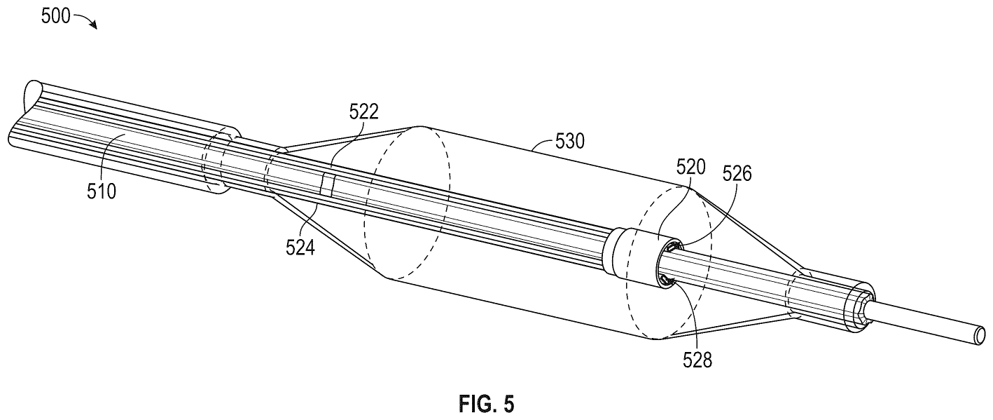

In some examples, the enclosure of a catheter does not include an opening. FIG. 5 illustrates an exemplary catheter 500 that does not include an opening in the enclosure 530 and can be used for catheter 10 in system 100. Such a catheter 500 may be referred to as a closed system catheter and may be generally similar to the catheters 10, 200, 300, and 400 described with respect to FIGS. 1, 2, 3, and 4 above. For example, the catheter 500 may include an elongated body 510, an enclosure 530, a first wire 522 and a second wire 524 extending along the elongated body 510, a conductive sheath 520 disposed around at least a portion of the elongated body 510. Optionally, the catheter 500 can include a guide wire 540. The conductive sheath 520, first wire 522, and second wire 524 may form one or more emitters, such as the first emitter 526 and a second emitter 528. In closed system catheters, such as the catheter 500 shown in FIG. 5, the emitters 526, 528 generate shock waves inside the closed volume of the enclosure 530 and cavitation bubbles formed at the emitters do not escape the enclosure 530. Advantageously, closed system catheters such as catheter 500 may permit the enclosure 530 to expand a greater amount than enclosures that include an opening. For instance, the enclosure 530 of the catheter 500 may be formed from relatively more flexible materials that are configured to expand a relatively greater amount then the enclosure of an open system catheter. In some examples, the enclosure 530 of a closed system catheter is an angioplasty balloon.

As described above, the elongated body of a catheter may include various lumens for carrying fluid, energy, and components of the catheter between a proximal portion of the catheter (e.g., the proximal handle 22 shown in FIG. 1) and a distal end of the catheter. FIG. 6 illustrates a cross-sectional view of the elongated body 610 of an exemplary catheter 600 showing the position of various lumens of the elongated body 610. The catheter 600 shown in FIG. 6 may be any of the exemplary catheters 10, 200, 300, 400, or 500 described above in relation to FIGS. 1, 2, 3, 4A-4B and 5 and include similar elements, such as an elongated body 610 and an enclosure 630.

The lumens of the elongated body 610 may be channels that extend longitudinally through the material of the elongated body 610. In some examples, the elongated body 610 includes a guide wire lumen 612 sized to carry a guide wire, such as a commercially available guide wire used for angioplasty procedures. The diameter of the guide wire lumen 612 may be slightly larger than the diameter of the guide wire to provide an additional tolerance that allows the elongated body 610 to slide easily along the guide wire without resistance. In a particular example, the guide wire lumen 612 is sized to receive a guide wire having a diameter of fourteen thousandths of an inch (0.014″), or about 0.035 mm. In some examples, the diameter of the guide wire lumen 612 is at least one hundred forty-one ten thousandths of an inch (0.0141″), or about 0.036 mm. However, the guide wire lumen 612 may be sized to receive guide wires having a larger or smaller diameter.

The elongated body 610 may further include one or more fluid lumens 614, 616 for flowing fluid into the enclosure 630 and evacuating the fluid from the enclosure 630. In some examples, the fluid lumens 614, 616 include an aspiration lumen (e.g., lumen 614) for flowing fluid into the enclosure 630 and a flush lumen (e.g., lumen 616) for evacuating fluid from the enclosure 630. Advantageously, flowing fluid through the enclosure 630 during a shock wave treatment may remove debris from the emitters inside the enclosure 630 and prevent accumulation of cavitation bubbles inside the enclosure 630. In some examples, fluid is flowed continuously through the enclosure 630 during a shock wave treatment.

In some examples, the elongated body 610 may include one or more lumens for carrying wires or optical fibers for providing energy to one or more emitters of the catheter 600. In some examples, one or more lumens described above may be combined, such that a single lumen serves the function of both lumens. For instance, a guide wire lumen 612 of the elongated body 610 may additionally carry fluid to or from the enclosure 630 or may additionally include one or more wires or optical fibers.

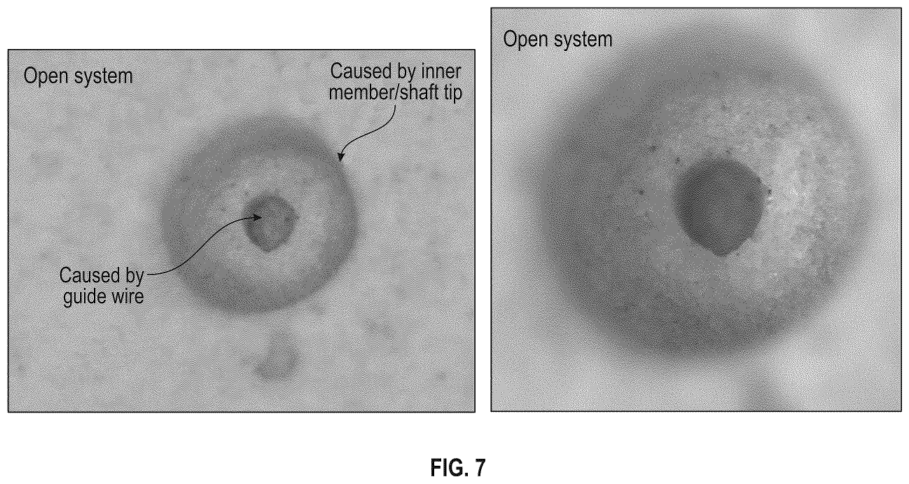

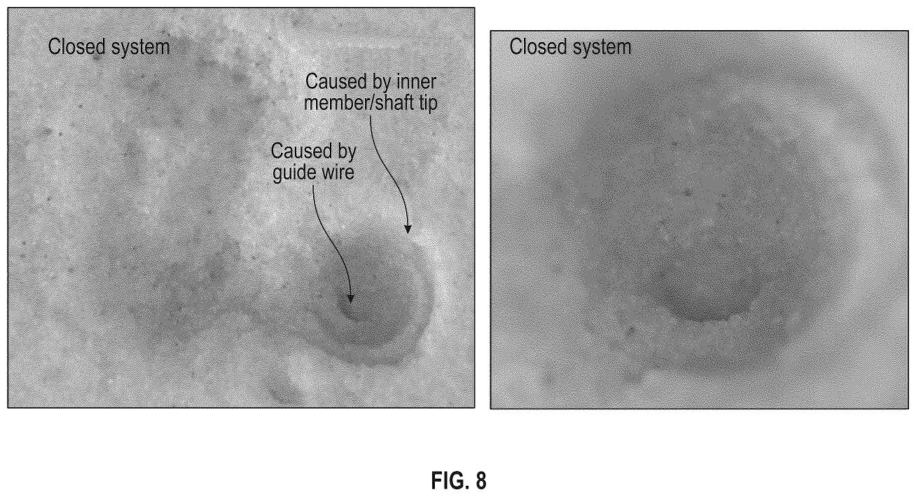

The effectiveness of such catheters for treating occlusions has been demonstrated by conducting experimental trials on a phantom surface made of a calcium mineral that mimics a calcified lesion. FIG. 7 illustrates the results of an experimental trial where an open-system catheter (e.g., the catheter 400 described above with respect to FIGS. 4A-4B) was used to treat a phantom surface made of the calcium mineral that mimics a calcified lesion. FIG. 8 illustrates the results of an experimental trial where a closed-system catheter (e.g., the catheter 500 described above with respect to FIG. 5) was used to treat a phantom surface made of the calcium mineral mimicking a calcified lesion. In the experimental trails, a guide wire was inserted into a guide wire lumen of the catheter and the guide wire remained inside the guide wire lumen during treatment, with at least a portion of the guide wire extending past the distal end of the catheter's elongated body. Shock waves were then generated at least one emitter of the catheters, causing the elongated body of the catheter to vibrate and the guide wire to vibrate in conjunction with the elongated body. When the vibrating guide wire and elongated body were placed in contact with the phantom surface, the guide wire and elongated body applied mechanical force to the phantom surface that caused holes to form in the phantom surface. As seen in FIGS. 7-8, the mechanical force of the guide wire produced a smaller diameter hole that penetrated deeper into the phantom surface. The elongated body (referred to in FIGS. 7-8 as the “inner member/shaft tip” produced a larger diameter hole that was shallower than the hole formed by the guide wire. Such trials demonstrate that mechanical forces applied by the vibrating guide wire and elongated body may be successful for penetrating and treating resistant occlusions in body lumens, such as fibrotic and calcified occlusions and chronic total occlusions (CTOs) in vasculature. Such trials additionally demonstrate that vibration of the guide wire may be used to initially puncture an occlusion prior to continuing the treatment of the occlusion with the wider-diameter elongated body.

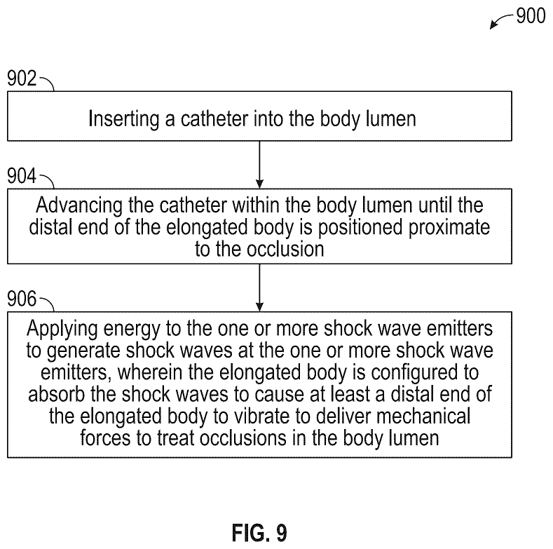

FIG. 9 illustrates a flowchart of an exemplary method 900 of treating an occlusion in a body lumen using a catheter, according to one or more examples of the present disclosure. Such a method may be performed using any of the catheters described herein, such as the catheters 10, 200, 300, 400, 500, or 600 described above with respect to FIGS. 1, 2, 3, 4A-4B, 5, and 6. As described above, such catheters may include at least a catheter enclosure, one or more shock wave emitters enclosed within the catheter enclosure, and an elongated body that extends to at least a distal end of the catheter enclosure.

In some examples, block 902 of the method 900 includes inserting a catheter into the body lumen. In some examples, the enclosure of the catheter is in a collapsed or folded state to reduce a crossing diameter of the catheter when the catheter is inserted into the body lumen. In some examples, insertion of the catheter into the body lumen is facilitated by a guide wire. For instance, introducing the catheter into the body lumen may include advancing the guide wire from an entry site on a patient (e.g., an artery in the groin area of the leg) to the target region of a vessel (e.g., a region having calcified plaques that need to be broken up), and advancing the catheter into the body lumen over the guide wire.

In some examples, block 904 of the method 900 includes advancing the catheter within the body lumen until the distal end of the elongated body is positioned proximate to the occlusion. In some examples, e.g., when a user intends to use the guide wire to treat the occlusion, the catheter may be advanced within the body lumen until a distal end of the guide wire is positioned proximate to the occlusion. In some examples, advancing the catheter through the body lumen includes advancing the catheter until a distal end of the elongated body and/guide wire is in contact with the occlusion. The location of the catheter (and/or guide wire) within the body lumen may be determined by x-ray imaging and/or fluoroscopy.

In some examples, when the catheter is positioned proximate to the occlusion, the method includes filling the enclosure with fluid (e.g., a conductive fluid such as saline and/or saline mixed with an image contrast agent). In some examples, filling the enclosure with conductive fluid causes the enclosure to inflate such that the outer surface of the enclosure contacts walls of the body lumen and/or lesions inside the body lumen. In some examples, filling the enclosure includes pressurizing the enclosure. For example, the enclosure may be pressurized to about 4 atm. In some examples, the enclosure may be pressurized in a closed system. In some examples, the enclosure may not be pressurized in an open system (i.e., an enclosure having an opening therein).

In some examples, block 906 of the method 900 includes applying energy to the one or more shock wave emitters to generate shock waves at the one or more shock wave emitters. The energy may be applied to the emitters by an external power source, such as the power source 28 shown in FIG. 1. In examples where the emitters include one or more electrode pairs, applying the energy may include applying a voltage to one or more of the shock wave emitters (e.g., by applying a voltage to one or more wires in electrical connection with an electrode pair of the emitter) by a voltage source. In examples where the emitters include one or more optical fibers, applying the energy may include applying laser energy by a laser energy source.

When the shock waves are generated at the emitters, the shock waves produce acoustic pressure waves that propagate through the fluid inside the enclosure. The elongated body of the catheter may be configured to vibrate based om at least a portion of the shock waves generated by the emitters. For example, the shock wave energy may force the distal end of the elongated body in a direction opposite the emitter(s), causing the distal end to move and vibrate inside the body lumen. As discussed above, vibration of the distal end of the elongated body may cause the guide wire to vibrate in conjunction with the distal end. In some examples, energy from the shock waves may propagate through the walls of the enclosure to treat lesions surrounding the enclosure. In some examples, generating shock waves produces cavitation bubbles inside the enclosure. In some examples, such as in an open system catheter, the generation of a shock wave may cause an opening in the enclosure of the catheter to open. Cavitation bubbles may then be directed outward through the opening towards an occlusion, where their collapse generates additional pressure to treat the lesion. Following propagation of a shock wave, the opening may return to a closed state. In other examples, such as in closed system catheters, shock waves are generated in a closed volume inside the enclosure.

In some examples, the method 900 includes generating a series of shock waves at one or more of the emitters to cause the elongated tube and/or guide wire to vibrate. For instance, a series of energy pulses (e.g., a series of voltage pulses or a series of laser pulses) may be applied by a power source to generate a series of shock waves at the emitters. In some examples, the series of energy pulses are applied at a frequency between 3-10 Hz, such as about, 4 Hz, 6 Hz, 8 Hz, etc. However, greater or lesser frequencies may also be used to generate shock waves during a shock wave treatment, and the number, magnitude, or frequency of the energy pulses may be controlled by a user of the catheter. In some examples, the power source may generate one or more bursts of micro-pulses in rapid succession (e.g., with a frequency between about 100 Hz-10 kHz). A series of the bursts of micro-pulses can be generated in accordance with the aforementioned frequency of about 3-10 Hz, such as between about 4-8 Hz. Generating several high-voltage pulses in a packet having a short duration (i.e., operating the one or more emitters in a “burst mode”) is described in greater detail in U.S. patent application Ser. No. 18/595,148, the contents of which are incorporated herein by reference in its entirety.

The method may additionally include maneuvering the catheter inside the body lumen to drive the vibrating guide wire and/or elongated body into the occlusion. For instance, the guide wire may extend more distally to the elongated body, and a user of the catheter may first cause a distal end of the guide wire to contact the occlusion and apply mechanical forces to the occlusion. Application of mechanical force with vibrating guide wire may cause an initial puncture of the occlusion (e.g., penetrate a fibrous cap of an occlusion). Further application of force may cause the guide wire to drill into the occlusion, creating a small hole in the occlusion. The user may then proceed to maneuver the distal end of the elongated body toward the lesion, causing the vibrating distal end to contact the occlusion and apply mechanical forces to the occlusion. In some examples, the distal end of the elongated body is used to treat a larger area of the occlusion proximate the initial puncture formed by the guide wire. However, as described above, in some examples the guide wire is removed from the catheter prior to initiating treatment. In such examples, the shock wave treatment may be performed by applying mechanical forces to the occlusion using the vibrating distal end of the elongated body to penetrate, disrupt, and clear the occlusion.

In some examples, the treatment may be conducted in one or more stages or phases. For instance, a physician may initially position the catheter near a first portion of an occlusion and apply mechanical forces to the first portion of the occlusion using the vibrating guide wire and/or elongated body. If the occlusion is not cleared by a first round of treatment, the user may reposition the catheter further along the length of the body lumen and treat a second portion of the occlusion using mechanical forces from the vibrating guide wire and/or elongated body. Once the occlusion has been sufficiently treated, the enclosure may optionally be inflated further or deflated, and the catheter and guide wire may be withdrawn from the patient.

Although the electrode assemblies and catheter devices described herein have been discussed primarily in the context of treating lesions in vasculature, such as chronic and resistant occlusions, the electrode assemblies and catheters herein can be used for a variety of occlusions, such as occlusions in the peripheral vasculature (e.g., above-the-knee, below-the-knee, iliac, carotid, etc.). For further examples, similar designs may be used for treating soft tissues, such as cancer and tumors (i.e., non-thermal ablation methods), blood clots, fibroids, cysts, organs, scar and fibrotic tissue removal, or other tissue destruction and removal. Emitter assembly and catheter designs could also be used for neurostimulation treatments, targeted drug delivery, treatments of tumors in body lumens (e.g., tumors in blood vessels, the esophagus, intestines, stomach, or vagina), wound treatment, non-surgical removal and destruction of tissue, or used in place of thermal treatments or cauterization for venous insufficiency and fallopian ligation (i.e., for permanent female contraception).

In one or more examples, the electrode assemblies and catheters described herein could also be used for tissue engineering methods, for instance, for mechanical tissue decellularization to create a bioactive scaffold in which new cells (e.g., exogenous or endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

The elements and features of the exemplary electrode assemblies and catheters discussed above may be rearranged, recombined, and modified, without departing from the present invention. Furthermore, numerical designators such as “first”, “second”, “third”, “fourth”, etc. are merely descriptive and do not indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged without departing from the subject invention.

It should be noted that the elements and features of the example catheters illustrated throughout this specification and drawings may be rearranged, recombined, and modified without departing from the present invention. For instance, while this specification and drawings describe and illustrate catheters having several enclosure and emitter designs, the present disclosure is intended to include catheters having a variety of enclosure and emitter configurations. The number, placement, and spacing of the shock wave emitters can be modified without departing from the subject invention. Further, the number, placement, and spacing of enclosures of catheters can be modified without departing from the subject invention.

It should be understood that the foregoing is only illustrative of the principles of the invention, and that various modifications, alterations and combinations can be made by those skilled in the art without departing from the scope and spirit of the invention. Any of the variations of the various catheters disclosed herein can include features described by any other catheters or combination of catheters herein. Furthermore, any of the methods can be used with any of the catheters disclosed. Accordingly, it is not intended that the invention be limited, except as by the appended claims.

1. A catheter for treating occlusions in a body lumen, the catheter comprising:
a catheter enclosure;
one or more shock wave emitters enclosed within the catheter enclosure; and
an elongated body that extends to at least a distal end of the catheter enclosure, wherein a distal end of the elongated body is configured to vibrate from shock waves produced by the shock wave emitters to deliver mechanical forces to an occlusion in the body lumen.

2. The catheter of claim 1, wherein the distal end of the elongated body extends distally past the distal end of the catheter enclosure.

3. The catheter of claim 1, wherein the distal end of the catheter enclosure is sealed to the distal end of the elongated body.

4. The catheter of claim 1, wherein the catheter enclosure comprises an angioplasty balloon.

5. The catheter of claim 4, wherein the catheter enclosure forms a closed volume around the elongated body.

6. The catheter of claim 1, wherein the catheter enclosure comprises an opening adjacent to one or more of the shock wave emitters.

7. The catheter of claim 6, wherein the opening is disposed in at least a tapered region of the catheter enclosure.

8. The catheter of claim 6, wherein the opening comprises a slit, the slit longitudinally aligned with a longitudinal axis of the elongated body.

9. The catheter of claim 1, wherein the elongated body comprises a guide wire lumen.

10. The catheter of claim 9, wherein the guide wire lumen is sized to receive a 0.014″ diameter guide wire.

11. The catheter of claim 10, wherein a diameter of the guide wire lumen is at least 0.0141″.

12. The catheter of claim 9, wherein vibration of the distal end of the elongated body causes a guide wire in the guide wire lumen to vibrate in conjunction with the distal end of the elongated body.

13. The catheter of claim 1, wherein the elongated body comprises a polymeric material.

14. The catheter of claim 1, wherein the elongated body comprises a first material, wherein the catheter enclosure comprises a second material, and wherein the first material is more rigid than the second material.

15. The catheter of claim 1, wherein the one or more shock wave emitters comprises an electrode pair.

16. The catheter of claim 15, wherein a first electrode of the electrode pair comprises a conductive sheath disposed around at least a portion of the elongated body.

17. The catheter of claim 16, wherein a second electrode of the electrode pair comprises a distal end of a conductive wire.

18. The catheter of claim 1, wherein the one or more shock wave emitters comprises an optical fiber.

19. The catheter of claim 1, wherein the one or more shock wave emitters comprises a first shock wave emitter and a second shock wave emitter.

20. The catheter of claim 19, wherein the second shock wave emitter is no greater than 90 degrees apart from the first shock wave emitter relative to a circumference of the elongated body.

21. The catheter of claim 19, wherein the second shock wave emitter is approximately 60 degrees apart from the first shock wave emitter relative to the circumference of the hollow tubular body.

22. A method of treating an occlusion in a body lumen, the method comprising:
inserting a catheter into the body lumen, the catheter comprising:
a catheter enclosure;
one or more shock wave emitters enclosed within the catheter enclosure; and
an elongated body that extends to at least a distal end of the catheter enclosure;
advancing the catheter within the body lumen until the distal end of the elongated body is positioned proximate to the occlusion; and
applying energy to the one or more shock wave emitters to generate shock waves at the one or more shock wave emitters, wherein a distal end of the elongated body is configured to vibrate from the shock waves to deliver mechanical forces to the occlusion.

23. The method of claim 22, wherein the catheter enclosure comprises an opening adjacent to one or more of the shock wave emitters.

24. The method of claim 23, wherein the opening is configured to open responsive to the generation of a shock wave, and wherein the opening is configured to close after termination of a shock wave.

25. The method of claim 24, wherein applying energy to the one or more shock wave emitters comprises applying a voltage to one or more of the shock wave emitters.

26. The method of claim 25, wherein applying energy to the one or more shock wave emitters comprises applying a series of voltage pulses to one or more of the shock wave emitters.

27. The method of claim 26, wherein the series of voltage pulses are applied at a frequency between 4 Hz and 8 Hz.

28. The method of claim 22, wherein applying energy to the one or more shock wave emitters comprises applying laser energy to one or more of the shock wave emitters.

29. The method of claim 22, wherein the elongated body comprises a guide wire lumen, and wherein inserting the catheter into the body lumen comprises:
inserting a guide wire into the body lumen; and
inserting the catheter into the body lumen over the guide wire.

30. The method of claim 29, wherein the vibration of the distal end of the elongated body causes the guide wire to vibrate in conjunction with the distal end of the elongated body such that the guide wire also delivers mechanical forces to treat the occlusion.

31. A system for treating occlusions in a body lumen, the system comprising:
a catheter comprising:
a catheter enclosure;
one or more shock wave emitters enclosed within the catheter enclosure; and
an elongated body that extends to at least a distal end of the catheter enclosure,
wherein a distal end of the elongated body is configured to vibrate from shock waves produced by the shock wave emitters to deliver mechanical forces to an occlusion in the body lumen; and
an energy generator configured to deliver energy to one or more of the shock wave emitters to generate the shock waves.

32. The system of claim 31, wherein the one or more shock wave emitters comprises one or more electrode pairs, and wherein the energy generator is configured to deliver a voltage to the one or more shock wave emitters.

33. The system of claim 31, wherein the one or more shock wave emitters comprises one or more optical fibers, and wherein the energy generator is configured to deliver laser energy to the one or more optical fibers.
